(19) United States
(12) Patent Application Publication
(10) Pub. No.: US 2011/0183403 A1
(43) Pub. Date: Jul. 28, 2011

(54) CELL DISRUPTION OF PLANT AND ANIMAL RAW MATERIALS BY A COMBINATION OF AUTOMIZATION PROCESS WITH DECOMPRESSION PROCESSES FOR SELECTIVE EXTRACTION AND SEPARATION OF INTERACELLULAR VALUABLE SUBSTANCES

(75) Inventors: Heribert Dierkes, Hagen (DE); Volkmar Steinhagen, Hagen (DE); Michael Bork, Dortmund (DE); Christoph Luetge, Unna (DE); Zeliko Knez, Maribor (SI)

(73) Assignee: UHDE HIGH PRESSURE TECHNOLOGIES GMBH, Hagen (DE)

(21) Appl. No.: 12/737,669

(22) PCT Filed: Aug. 6, 2009
(86) PCT No.: PCT/EP2009/005689

§ 371 Date: Apr. 1, 2011

(30) Foreign Application Priority Data
Aug. 7, 2008 (DE) 102008036723.0

(51) Int. Cl. C12N 1/12

(52) U.S. Cl. 435/257.1

(57) ABSTRACT

A method for cell disruption of biogenic suspended raw materials by means of a combination of pressurization, atomization and decompression with a subsequent selective extraction and separation of cellular valuable substances includes at least one reservoir cubicle serving as reservoir for a suspension composed of biogenic raw material and at least another reservoir cubicle utilized as reservoir for a solvent. A cellular extract is produced in one unit for cell disruption, and is subsequently flown through by a gas in an extraction stage. The gas burdened with cellular valuable substances is separated from the cellular valuable substances in a separation stage by lowering the pressure. The suspension includes biogenic raw material pressurized to a pressure of 100-2500 bar by a device for pressure boosting. The solvent is pressurized to a pressure of 100-2500 bar by a device for pressure boosting. The solvent and the suspension are brought together in one line at a pressure of 100-2500 bar and mixed to a solution mixture which is atomized through at least one jet at a pressure of 100 to 2500 bar and a temperature of 10 to 90° C. into a cubicle with a lower pressure.

The invention relates to a method for cell disruption of plant or animal raw materials by means of atomization processes in combination with a decompression of cells for subsequent selective extraction and separation of constituents contained therein.

Mechanical cell disruption methods known in this connection are the method of homogenizing with rotating knife edges which in most cases is performed at high pressure, disruption in a stirring mill, pressing of a sample at high pressure through a narrow aperture or a method applied by means of an ultrasonic homogenizer.

The drawback of these mechanical methods is the shear forces acting on the cells due to friction which when trying to overcome them at high velocities lead to a formation of heat and consequently generate a high temperature. This may entail a harmful impact on the constituents of the evolving cellular extracts.

A more gentle method is provided by a physical decompression of cells. According to Henry's law, suspension gas in cells is enriched at higher gas pressures and the cell membranes are thus caused to burst due to an abrupt pressure relief. This occurs because the dissolved gas cannot escape fast enough and bubbles out within the cells in form of growing gas bubbles. As a result, the mechanical load acting on the cell rises until the cell bursts and the cell contents is released.

A major drawback of the decompression method lies in that only cells that are relatively easy to break up can be efficiently disrupted which calls for additional application of non-mechanical disruption processes like the use of enzymes. This in turn renders a disruption process non-profitable for large-scale applications.

According to the state-of-the-art technology, extraction is executed by means of supercritical fluids. This term relates to gases or liquids which are above their critical temperature and critical pressure that are defined in the relevant phase diagram of a pure substance. The benefit lies in an increased solubility for hardly soluble substances in the supercritical range. Moreover, solubility can still be controlled via alterations in pressure or temperature. An example is given by the decaffeination of a tea plant by means of supercritical CO₂ as described in WO 2008/05537 A1. But other applications in chemical industry and foodstuff industry have meanwhile become well established methods, for example the extraction of oils, ginger, black pepper or chili powder by means of supercritical CO₂ or supercritical propane.

The publication WO 2008/061716 A1 describes a further improvement in the method of extraction in which entraining agents usually applied to ensure a further increase in solubility without any further increase in pressure are dispensed with.

EP 0 941 140 B1 describes the extraction of various products from a fermentation medium by means of carbon dioxide of supercritical or almost critical state. The extraction described in this patent is based on a water-based suspension. There is no indication of a simultaneous cell disruption and the extraction of the substances. As the solvent of supercritical or almost critical state is carbon dioxide which has a relatively good solubility in water, extracts of high water content are expected to be obtained. Moreover, the extraction temperatures selected are relatively high as a result of which different hydrolytic reactions of the extracted substances with water can take place.

U.S. Pat. No. 5,306,637 describes a method for cell disruption in which an enzymatic disruption is linked to a subsequent abrupt pressure relief causing the cells to burst and effectively releasing the valuable substance contained in them. However, the pressures applied are low such that a prolonged saturation period is to be taken into account. This invention uses carbon dioxide, which is of supercritical or almost supercritical state and to which entraining agents such as sulphur oxide, nitrogen monoxide, hydrogen peroxide, ethanol or mixtures of these entraining agents are optionally added, for the disruption of microbial cells and for the extraction of intracellular components such as proteins or nucleic acids. The isolation of proteins or nucleic acids is not based on the solubility of these substances in the gases used but on the precipitation of these substances from the suspension. Here, the recovery of the gas mixtures is extremely problematic and, therefore, the process described is not applied on an industrial scale.

In WO91/01367 a solvent is selected first which is in the form of a gas and has a critical temperature between 0 and 100° C. This solvent is brought to a pressure near or even higher than the critical pressure of the respective solvent and to a temperature near the critical temperature of the respective solvent. The solvent is then brought together with a suspension of the cellular material in order to saturate the cells with the solvent under the specified conditions. In the next step the pressure is lowered resulting in a partial destruction of the cell membrane and the release of cellular components. Then the disrupted cell material is introduced into a second boiler. However, as known from the state of the art it is to be expected that the released proteins and nucleic acids to be dissolved in this way have an extremely low solubility in solvents of supercritical or almost critical state.

This means that, despite the permanent improvement of the state-of-the-art methods for cell disruption, there still remain substantial residues of the substance to be isolated in the cellular extract.

Hence there still is the need to achieve the greatest possible yield and purity of the desired hardly soluble intracellular valuable substances from the cellular extract in a supercritical solvent or a solvent by improvements in the existing processes, which the invention to be described here takes charge of.

This task is solved by means of the inventive method for cell disruption of biogenic suspended raw materials by means of a combination of pressurization, atomization and decompression including a subsequent selective extraction and separation of cellular valuable substances, with at least one reservoir cubicle serving as reservoir for a suspension composed of biogenic raw material and with at least another reservoir cubicle being utilized as reservoir for a solvent, and wherein a cellular extract is produced in one unit for cell disruption, and wherein the cellular extract is subsequently flown through by a gas in an extraction stage and wherein the gas burdened with cellular valuable substances is separated in a separation stage from the cellular valuable substances by lowering the pressure. The suspension of biogenic raw material is pressurized to 100 to 2500 bar by a device for pressure boosting, and the solvent is also pressurized to 100 to 2500 bar by a device for pressure boosting; subsequently the solvent and the suspension are brought together in one line at a pressure of 100 to 2500 bar and mixed to a solution mixture, and then the solution mixture is atomized through at least one jet at a pressure of 100 to 2500 bar and a temperature of 10 to 90° C. into a cubicle with a lower pressure. By this method it is achieved that the cell disruption of the biogenic suspended raw material and the dissolution of the cellular valuable substances are performed simultaneously.

In general, cell disruption is improved the higher the selected pressure of the solution mixture composed of biogenic suspended cellular material and solvent. In view of equipment costs and for process technology-related considerations, it is most advantageous to stay within a pressure range between 1300 and 1600 bar.

In one embodiment of the method, the solvent with which the suspension of biogenic raw material is mixed is selected from a group that contains ethane, ethylene, propane, propylene, butane, butylene, other saturated or unsaturated hydrocarbons, carbon dioxide, nitrous oxide, dimethyl ether, sulphur hexafluoride, R 134a, R125, R32, R141b, freons and mixtures thereof. Optionally, the solvent with which the suspension of biogenic raw material is mixed is a supercritical fluid that is not a hydrocarbon. The solvent of supercritical or almost critical state is characterized in that water is virtually insoluble in it.

Optionally, the suspension composed of biogenic raw material can be saturated or oversaturated with the solvent prior to atomization.

In another advantageous embodiment of the method, the biogenic raw material is pre-treated by scrubbing, filtering, crushing, grinding or screening prior to the formation of the suspension.

Further possibilities to configure the inventive method for cell disruption of biogenic suspended raw material by means of pressurization, atomization and decompression relate to a combination with other disruption methods. Accordingly, these disruption methods are either selected from a group of mechanical processes that contains cell disruption by means of a high-pressure homogenizer, ball mill, ultrasonic homogenizer, French press and impact blast apparatuses. Alternatively, these disruption processes are selected from a group of chemical methods that contains cell disruption by means of antibiotics, chelate forming agents, chaotropic agents, detergents, and alkaline treatment. Accordingly, the cell walls are either permeabilized or saponified. Furthermore, it is possible to link the inventive method with a disruption method which is selected from a group of biological methods that contains cell disruption by means of enzymes, phages or autolysis. Optionally, this method can be selected from a group of physical methods that contains cell disruption by means of freezing and thawing, thermolysis or decompression.

Optionally the applied biogenic suspended raw material is composed of algae.

Further possibilities to configure this method relate to the cellular valuable substances to be extracted. On the one hand, these are cellular valuable substances of the class of carotenoids, like carotenes or xanthophylls. Alternatively, the cellular valuable substance to be extracted is astaxanthin. Furthermore eligible is an extraction of cellular valuable substances from the substance class of fats and oils that are contained in the biogenic suspended raw material.

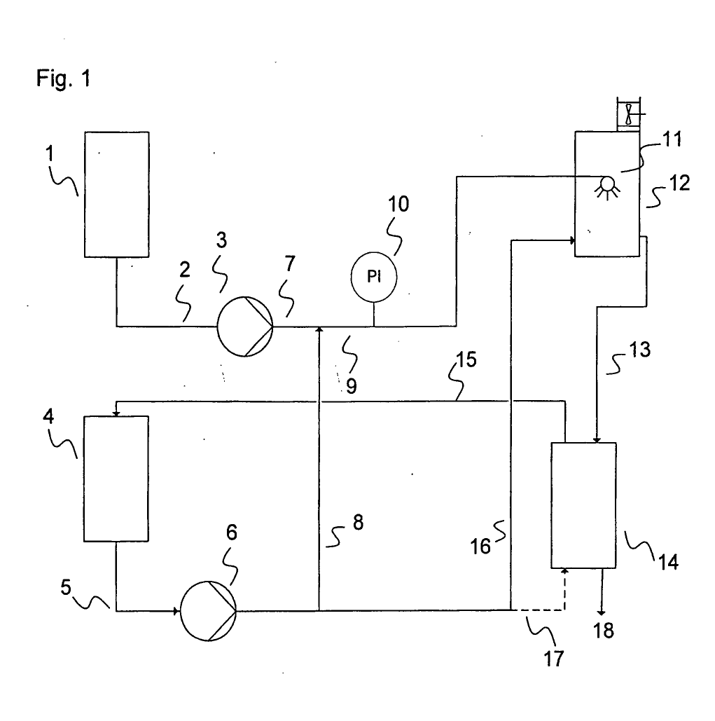

An exemplary embodiment of the present invention is illustrated in FIG. 1 and explained in greater detail as set forth below, where:

FIG. 1: shows an inventive process sketch of the method for cell disruption of biogenic suspended raw materials by means of a combination of pressurization, atomization and decompression with a subsequent selective extraction and separation of cellular valuable substances.

FIG. 1 shows a reservoir cubicle 1 to take-up the biogenic suspended raw material as well as another reservoir cubicle 4 to take-up the solvent. The biogenic suspended raw material 2 is pressurized by pump 3 to a pressure ranging between 100 and 2500 bar. The solvent 5 is also pressurized by a pump 6 to a pressure ranging between 100 and 2500 bar. Accordingly, the biogenic suspended raw material 2 and the solvent 5 can be pressurized to the same pressure or, alternatively, the pressures may differ from each other. Subsequently the pressurized biogenic raw material 7 is brought together with the pressurized solvent 8 in one line to obtain a solution mixture 9 that has also been pressurized to a pressure ranging between 100 and 2500 bar. This line pressure is controlled via a pressure gauge 10. The solution mixture 9 is then introduced into a cubicle 12 which has a lower pressure than the solution mixture, with the introduction of the solution mixture being accomplished through jets 11 that inject the solution mixture 9 into the cubicle 12. Thus, the pressure reduction and the atomization process of the solution mixture 9 are performed simultaneously. A solvent for extraction 16 and supplied in counter-current to the solution mixture 9 is fed to the cubicle 12. Here, the solvent for extraction 16 is supplied from the reservoir cubicle 4 and thus corresponds to the one which is used for cell disruption by a combination of atomization processes and decompression. The extraction itself is performed according to standard state-of-the-art conditions. The extracted substances including the solvent are conveyed by stream 13 to a cubicle 14 serving as a separator where the extracted substances are separated from the solvent after pressure reduction. The solvent thereby recovered is again passed via a return line 15 into the reservoir cubicle 4 for take-up of the solvent and the extracted substances are removed from the cubicle 14 as stream 18.

Alternatively, the solvent 17 for extraction is conveyed to the cubicle 14 serving in this case as extraction cubicle where the solvent is conveyed in counter-current to stream 13 which in this case is the cell disruption material from the cubicle 12. In this, the extraction is performed according to standard state-of-the-art conditions. For extraction the pressure is maintained at the same level as the pressure prevailing in the cubicle 12. With this process variant an additional cubicle is required which serves as a separator (not shown in FIG. 1).

The process parameters to be observed here are optimum with the use of CO₂ as solvent 5 in a pressure range between 300 and 2500 bar, in a temperature range between 10° C. and 90° C. and in a ratio of solvent 5 to biogenic suspended raw material 2 between 5 and 90 kg/kg. If the process is run using C2 to C4 hydrocarbons, the pressure range can be set to values between 100 and 2500 bar, the ratio of solvent 5 to biogenic suspended raw material 2 having to be limited to between 1 and 60 kg/kg. The process can be run both batchwise and continuously.

By the process conditions thus defined and characterized by a very high pressure, low temperatures and an optionally continuous process mode it is possible to extract the desired cellular valuable substances of high concentration and high purity.

The inventive method is to be delimited from the state of the art as described, for example, in WO91/01367A1 by two examples.

### EXAMPLE 1

A. Inventive Method:
1 kg of aqueous suspension of the Hematococcus pluvialis alga which has a solids concentration of 181.1 g/kg and an oil content of approx. 13.7 wt. % (referred to the dry weight) is brought to a pressure of 1500 bar by means of a pump and then mixed with the solvent CO₂ under pressure. The solution mixture thereby obtained is atomized through a jet into a pressure vessel with a lower pressure of 300 bar. The combined decompression and atomization process achieves the disruption of the algae in the solution mixture and the fine atomization of the solution mixture in the pressure vessel. The atomized solution mixture including the disrupted algae is then extracted in the pressure vessel in counter-current with the solvent CO₂ without a change in pressure at 300 bar. The extracted substances are conveyed together with the CO₂ solvent to a separator where the extracted substances are separated from the solvent after pressure reduction. In this example, the ratio of CO₂ solvent to biogenic suspended raw material is 90 kg/kg.
The product obtained in this case is approx. 0.27 kg and consists of an oil/water emulsion, the oil-containing phase being separated by sedimentation. This results in a total amount of extracted oil of 20.1 g/kg algae suspension.

B. State-of-the-Art Method:
1 kg algae suspension of the Hematococcus pluvialis alga which has a solids concentration of 181.1 g/kg and an oil content of approx. 13.7 wt. % (referred to the dry weight) is brought to a pressure of 1500 bar by means of a pump and then mixed with the solvent CO₂ under pressure. The solution mixture thereby obtained is depressurized to atmospheric pressure through a jet into a vessel but is not finely atomized. The decompression causes the disruption of the algae in the solution mixture. Then the disrupted algae suspension is conveyed to a pressure vessel at a pressure of 300 bar where it is atomized. The atomized solution mixture including the disrupted algae is extracted in counter-current with the solution mixture. Hence, with this method, decompression and atomization take place in two successive process steps and not simultaneously. The extracted substances are then conveyed together with the solvent to a separator where the extracted substances are separated from the solvent after pressure reduction. In this example, the ratio of CO₂ solvent to biogenic suspended raw material is 110 kg/kg. The product obtained in this case is approx. 0.20 kg and consists of an oil/water emulsion, with the oil-containing phase being separated by sedimentation. This results in a total amount of extracted oil of 15.8 g/kg algae suspension.

The results obtained according to example 1 can be interpreted such that the product yield could be increased by 21.4% as a result of the inventive method.

### EXAMPLE 2

A. Inventive Method:
1 kg aqueous suspension of the Hematococcus pluvialis alga which has a solids concentration of 181.1 g/kg and an oil content of approx. 13.7 wt. % (referred to the dry weight) is brought to a pressure of 1000 bar by means of a pump and then mixed with the solvent propane under pressure. The solution mixture thereby obtained is atomized through a jet into a pressure vessel with a lower pressure of 50 bar. The combined decompression and atomization process achieves the disruption of the algae in the solution mixture and the fine atomization of the solution mixture in the pressure vessel. The atomized solution mixture including the disrupted algae is then extracted in counter-current with the propane solvent in the pressure vessel without a change in pressure at 50 bar. The extracted substances are conveyed together with the solvent to a separator where the extracted substances are separated from the solvent after pressure reduction. In this example, the ratio of propane solvent to biogenic suspended raw material is 8 kg/kg.
The product obtained in this case consists of almost pure oil. The total amount of extracted oil is 22.1 g/kg algae suspension.

B. State-of-the-Art Method:
1 kg aqueous algae suspension of the Hematococcus pluvialis alga which has a solids concentration of 181.1 g/kg and an oil content of approx. 13.7 wt. % (referred to the dry weight) is brought to a pressure of 1000 bar by means of a pump and then mixed with the solvent propane under pressure. The solution mixture thereby obtained is depressurized to atmospheric pressure through a jet into a vessel but is not finely atomized. The decompression causes the disruption of the algae in the solution mixture. Then the disrupted algae suspension is conveyed to a pressure vessel at a pressure of 50 bar where it is atomized. The atomized solution mixture including the disrupted algae is extracted in counter-current with the solution mixture. Hence, with this method, decompression and atomization take place in two successive process steps and not simultaneously. The extracted substances are then conveyed together with the solvent to a separator where the extracted substances are separated from the solvent after pressure reduction. In this example, the ratio of propane solvent to biogenic suspended raw material is 11 kg/kg. The product obtained in this case consists of almost pure oil. The total amount of extracted oil is 18.8 g/kg algae suspension.

The results obtained according to example 2 can be interpreted such that the product yield could be increased by 15% as a result of the inventive method.

### EXAMPLE 3

A. Inventive Method:
0.9 kg aqueous suspension of the Hematococcus pluvialis alga which has a solids concentration of 181.1 g/kg and an astaxanthin content of approx. 3.0 wt. % (referred to the dry weight) is brought to a pressure of 2400 bar by means of a pump and then mixed with the solvent CO₂ under pressure. The solution mixture thereby obtained is atomized through a jet into a pressure vessel with a lower pressure of 500 bar. The combined decompression and atomization process achieves the disruption of the algae in the solution mixture and the fine atomization of the solution mixture in the pressure vessel. The atomized solution mixture including the disrupted algae is then extracted in counter-current with the CO₂ solvent in the pressure vessel without a change in pressure at 500 bar. The extracted substances are conveyed together with the CO₂ solvent to a separator where the extracted substances are separated from the solvent after pressure reduction. In this example, the ratio of CO₂ solvent to biogenic suspended raw material is 88 kg/kg.
The product obtained in this case is approx. 0.29 kg and consists of an oil/astaxanthin/water emulsion, the oil-containing phase being separated by sedimentation. Subsequently, a yield of 88.1% astaxanthin referred to the total astaxanthin content of the algae material used can be isolated from the oil-containing phase.

B. State-of-the-Art Method:
0.9 kg aqueous algae suspension of the Hematococcus pluvialis alga which has a solids concentration of 181.1 g/kg and an astaxanthin content of approx. 3.0 wt. % (referred to the dry weight) is brought to a pressure of 2400 bar by means of a pump and then mixed with the solvent CO₂ under pressure. The solution mixture thereby obtained is depressurized to atmospheric pressure through a jet into a vessel but is not finely atomized. The decompression achieves the disruption of the algae in the solution mixture. Then the disrupted algae suspension is conveyed to a pressure vessel at a pressure of 500 bar where it is atomized. The atomized solution mixture including the disrupted algae is extracted in counter-current with the solution mixture. Hence, with this method, decompression and atomization take place in two successive process steps and not simultaneously. The extracted substances are then conveyed together with the solvent to a separator where the extracted substances are separated from the solvent after pressure reduction. In this example, the ratio of CO₂ solvent to biogenic suspended raw material is 115 kg/kg. The product obtained in this case is approx. 0.22 kg and consists of an oil/astaxanthin/water emulsion, the oil-containing phase being separated by sedimentation. Subsequently, a yield of 72.3% astaxanthin referred to the total astaxanthin content of the algae material used can be isolated from the oil-containing phase.

The results obtained according to example 3 can be interpreted such that the product yield could be increased by 21.8% as a result of the inventive method.

### EXAMPLE 4

A. Inventive Method:
0.9 kg aqueous suspension of the Hematococcus pluvialis alga which has a solids concentration of 181.1 g/kg and an astaxanthin content of approx. 3.0 wt. % (referred to the dry weight) is brought to a pressure of 1600 bar by means of a pump and then mixed with the solvent butane under pressure. The solution mixture thereby obtained is atomized through a jet into a pressure vessel with a lower pressure of 150 bar. The combined decompression and atomization process achieves the disruption of the algae in the solution mixture and the fine atomization of the solution mixture in the pressure vessel. The atomized solution mixture including the disrupted algae is then extracted in counter-current with the butane solvent in the pressure vessel without a change in pressure at 150 bar. The extracted substances are conveyed together with the butane solvent to a separator where the extracted substances are separated from the solvent after pressure reduction. In this example, the ratio of solvent butane to biogenic suspended raw material is 8 kg/kg. The product obtained in this case is approx. 26.1 g and consists of oil and astaxanthin. Subsequently, a yield of 92.6% astaxanthin referred to the total astaxanthin content of the algae material used can be isolated from the oil-containing phase.

B. State-of-the-Art Method:
0.9 kg aqueous algae suspension of the Hematococcus pluvialis alga which has a solids concentration of 181.1 g/kg and an astaxanthin content of approx. 3.0 wt. % (referred to the dry weight) is brought to a pressure of 1600 bar by means of a pump and then mixed with the solvent butane under pressure. The solution mixture thereby obtained is depressurized to atmospheric pressure through a jet into a vessel but is not finely atomized. The decompression causes the disruption of the algae in the solution mixture. Then the disrupted algae suspension is conveyed to a pressure vessel at a pressure of 150 bar where it is atomized. The atomized solution mixture including the disrupted algae is extracted in counter-current with the solution mixture. Hence, with this method, decompression and atomization take place in two successive process steps and not simultaneously. The extracted substances are then conveyed together with the solvent to a separator where the extracted substances are separated from the solvent after pressure reduction. In this example, the ratio of butane solvent to biogenic suspended raw material is 11 kg/kg.
The product obtained in this case is approx. 22.0 g and consists of oil and astaxanthin. Subsequently, a yield of 78.4% astaxanthin referred to the total astaxanthin content of the algae material used can be isolated from the oil-containing phase.

The results obtained according to example 4 can be interpreted such that the product yield could be increased by 18.1% as a result of the inventive method.

Benefits Resulting from the Invention:
It is a gentle method of cell disruption because the chemical and physical loads acting on the inner cellular constituents are only small.
Increase in the degree of disruption of samples up to and including a disruption of hardly accessible subcellular organelles like cell cores or mitrochondrions.
Uniform degree of homogenization after cell disruption facilitates subsequent extraction.
Extensive drying of raw material can be dispensed with.
High concentration and high purity of the desired cellular valuable substances.

### LIST OF REFERENCE SYMBOLS

1 Reservoir cubicle for take-up of biogenic suspended raw material
2 Biogenic suspended raw material
3 Pump
4 Reservoir cubicle
5 Solvent
6 Pump
7 Pressurized biogenic suspended raw material
8 Pressurized solvent
9 Solution mixture
10 Pressure gauge
11 Injection jets
12 Cubicle
13 Stream
14 Cubicle
15 Return line
16 Solvent for extraction
17 Solvent
18 Stream
19 Extracted substances

1-16. (canceled)

17. A method for cell disruption of biogenic suspended raw materials by means of a combination of pressurization, atomization and decompression with a subsequent selective extraction and separation of cellular valuable substances, comprising:
providing at least one reservoir cubicle serving as a reservoir for a suspension composed of biogenic raw material;
providing at least another reservoir cubicle utilized as a reservoir for a solvent;
producing a cellular extract in one unit for cell disruption;
subsequently passing a gas through the cellular extract in an extraction stage;
separating the gas burdened with cellular valuable substances from the cellular valuable substances in a separation stage by lowering the pressure;
pressurizing the suspension composed of biogenic raw material to a pressure of 100-2500 bar by a device for pressure boosting;
pressurizing the solvent to a pressure of 100-2500 bar by a device for pressure boosting;
bringing together the solvent and the suspension in one line at a pressure of 100-2500 bar and mixed to a solution mixture; and
atomizing the solution mixture through at least one jet at a pressure of 100 to 2500 bar and a temperature of 10 to 90° C. into a cubicle with a lower pressure.

18. The method according to claim 17, wherein the solvent with which the suspension of biogenic raw material is mixed is selected from the group consisting of: ethane, propane, butane, carbon dioxide, nitrous oxide, ethylene, propylene, butylene, other saturated or unsaturated hydrocarbons, dimethyl ether, sulphur hexafluoride, R 134a, R125, R32, R141b, freons and mixtures thereof.

19. The method according to claim 17, wherein the solvent with which the suspension composed of biogenic raw material is mixed is a supercritical fluid that is not a hydrocarbon.

20. The method according to claim 17, wherein the suspension composed of biogenic raw material is saturated with the solvent prior to atomization.

21. The method according to claim 17, wherein the suspension composed of biogenic raw material is oversaturated with the solvent prior to atomization.

22. The method according to claim 17, wherein the applied solvent is recovered and returned into the reservoir cubicle for solvents.

23. The method according to claim 17, wherein the biogenic raw material is pretreated by scrubbing, filtering, crushing, grinding or screening prior to the formation of the suspension.

24. The method according to claim 17, wherein the method for cell disruption of biogenic suspended raw material by means of a combination of pressurization, atomization and decompression is linked to other disruption methods that are selected from a group of mechanical methods that contains cell disruption by means of a high-pressure homogenizer, ball mill, ultrasonic homogenizer, French press and impact blast apparatuses.

25. The method according to claim 17, wherein the method for cell disruption of biogenic suspended raw material by means of a combination of pressurization, atomization and decompression is linked to other disruption methods that are selected from a group of chemical methods that contains cell disruption by means of antibiotics, chelate forming agents, chaotropic agents, detergents, and alkaline treatment.

26. The method according to claim 17, wherein the method for cell disruption of biogenic suspended raw material by means of a combination of pressurization, atomization and decompression is linked to other disruption methods that are selected from a group of biological methods that contains cell disruption by means of enzymes, phages, or autolysis.

27. The method according to claim 17, wherein the method for cell disruption of biogenic suspended raw material by means of a combination of pressurization, atomization and decompression is linked to other disruption methods that are selected from a group of physical methods that contains cell disruption by means of freezing and thawing, thermolysis or decompression.

28. The method according to claim 17, wherein the applied biogenic suspended raw material is algae.

29. The method according to claim 17, wherein cellular valuable substances of the class of carotenoids, like carotenes or xanthophylls, contained in the biogenic suspended raw material are extracted.

30. The method according to claim 17, wherein the cellular valuable substance astaxanthin contained in the biogenic suspended raw material is extracted.

31. The method according to claim 17, wherein cellular valuable substances from the substance class of fats and oils contained in the biogenic suspended raw material are extracted.

32. The method according to claim 17, wherein the ratio of solvent to biogenic suspended raw material is between 1 and 90 kg/kg.
